(19) United States
(12) Patent Application Publication
(10) Pub. No.: US 2021/0161419 A1
(43) Pub. Date: Jun. 3, 2021

(54) COMPONENT CONCENTRATION MEASUREMENT DEVICE AND COMPONENT CONCENTRATION MEASUREMENT METHOD

(71) Applicant: Nippon Telegraph and Telephone Corporation, Tokyo (JP)

(72) Inventors: Masahito Nakamura, Tokyo (JP); Takuro Tajima, Tokyo (JP); Michiko Seyama, Tokyo (JP)

(21) Appl. No.: 17/048,454

(22) PCT Filed: Apr. 12, 2019
(86) PCT No.: PCT/JP2019/015964

§ 371 Date: Oct. 16, 2020

(30) Foreign Application Priority Data
Apr. 20, 2018 (JP) 2018-081176

(51) Int. Cl. A61B 5/05; A61B 5/145; A61B 5/1495

(52) U.S. Cl. CPC A61B 5/05; A61B 5/0507; A61B 5/1495; A61B 5/14532

(57) ABSTRACT

A component concentration measuring apparatus includes: a dielectric spectroscopy portion that irradiates a measurement subject with electromagnetic waves, thereby acquiring a complex permittivity spectrum; a signal processing portion that standardizes an imaginary part or an imaginary part spectrum of a complex permittivity at a frequency other than a frequency of an isosbestic point of the measurement subject, using an imaginary part of a complex permittivity at the frequency of the isosbestic point, out of the complex permittivity spectrum; and a calculating portion that applies a calibration model generated in advance from an imaginary part or an imaginary part spectrum of a complex permittivity of a sample whose component concentration is known, to the imaginary part or the imaginary part spectrum of the complex permittivity standardized by the signal processing portion, thereby calculating a component concentration of the measurement subject.

### CROSS-REFERENCE TO RELATED APPLICATIONS

This application is a national phase entry of PCT Application No. PCT/JP2019/015964, filed on Apr. 12, 2019, which claims priority to Japanese Application No. 2018-081176, filed on Apr. 20, 2018, which applications are hereby incorporated herein by reference.

### TECHNICAL FIELD

The present invention relates to a technique for measuring a component concentration of a component of interest, using the dielectric spectroscopic technique.

### BACKGROUND

Recently, demand is on the rise for wearable terminals in the health care field, and development of techniques for measuring various types of medical information with ease is in demand. As the measurement subject, blood components such as a blood glucose level, a water content of the skin, and the like are conceivable. For example, tests of a blood glucose level and the like involve drawing blood, and thus they significantly stress patients. Thus, non-invasive component concentration measuring methods not involving drawing blood have been gaining attention.

As non-invasive component concentration measuring methods, some methods using electromagnetic waves in microwave to millimeter-wave bands have been proposed because scattering is unlikely to occur in a living body compared with optical techniques using near infrared light or the like, and the energy in one photon is low, for example. For example, in the method disclosed in NPL 1, frequency characteristics around the resonance frequency are measured by bringing a device with a high Q factor such as an antenna or a resonator into contact with a sample that is to be measured. The resonance frequency is determined by a complex permittivity around a device, and thus, according to methods for measuring the shift amount of the resonance frequency, a correlation between shift amounts and component concentrations is measured in advance, and a component concentration is estimated from a shift amount of a resonance frequency.

As another component concentration measuring method using electromagnetic waves in microwave to millimeter-wave bands, a dielectric spectroscopic technique has been proposed (PL 1). According to the dielectric spectroscopic technique, the subcutaneous part is irradiated with electromagnetic waves, electromagnetic waves are allowed to be absorbed according to the interaction between a blood component that is a measurement subject, such as a glucose molecule, and water, and the amplitude and the phase of electromagnetic waves are observed. A dielectric relaxation spectrum is calculated from the amplitude or the phase of a signal corresponding to the frequency of observed electromagnetic waves. Typically, a dielectric relaxation spectrum is expressed in the form of linear combination of relaxation curves based on the Cole-Cole plot, and is used to calculate complex permittivity. The complex permittivity has a correlation, for example, with the amount of blood component such as glucose or cholesterol contained in blood in measurement of biological components, and is measured as an electrical signal (amplitude, phase) corresponding to a change thereof. A calibration model is constructed by measuring in advance a correlation between changes in complex permittivity and component concentrations, and calibration of the component concentration is performed from a change in the measured dielectric relaxation spectrum.

The dielectric spectroscopic technique measures a spectrum obtained by overlapping spectra unique to substances, and thus a feature amount unique to a measurement subject can be extracted using a statistical multivariate analysis method. Accordingly, this technique is superior to the resonator technique disclosed in NPL 1, regarding component concentration measurement in a multi-component system such as a blood system.

Furthermore, it is also possible to measure the water content in a living body, by performing component analysis regarding water using the dielectric spectroscopic technique, that is, dielectric spectroscopy is a technique that can be applied to both of component analysis and water content measurement.

However, in component concentration measurement using the dielectric spectroscopic technique, a change in a spectrum of hydrated water is observed, and thus this method is problematic in that, when the water content of a measurement subject changes, the dielectric spectroscopy spectrum changes in accordance with the change in the water content, which leads to a decrease in the level of measurement precision.

### CITATION LIST

### Patent Literature

PTL 1 Japanese Patent Application Publication No. 2016-188778

### Non Patent Literature

NPL 1—G. Guarin, M. Hofmann, J. Nehring, R. Weigel, G. Fischer, and D. Kissinger, “Miniature Microwave Biosensors”, IEEE Microwave Magazine, May 2015, pp. 71-86.

### SUMMARY

### Summary of Embodiments of the Invention

### Technical Problem

With the foregoing in view, it is an object of embodiments of the present invention to make it possible to measure the component concentration at a high level of precision, by suppressing the influence of the water content of a measurement subject when measuring the component concentration using the dielectric spectroscopy.

### Means for Solving the Problem

Embodiments of the present invention are directed to a component concentration measuring apparatus including: a dielectric spectroscopy portion that irradiates a measurement subject with electromagnetic waves, thereby acquiring a complex permittivity spectrum; a signal processing portion that standardizes an imaginary part or an imaginary part spectrum of a complex permittivity at a frequency other than a frequency of an isosbestic point of the measurement subject, using an imaginary part of a complex permittivity at the frequency of the isosbestic point, out of the complex permittivity spectrum; and a calculating portion that applies a calibration model generated in advance from an imaginary part or an imaginary part spectrum of a complex permittivity of a sample whose component concentration is known, to the imaginary part or the imaginary part spectrum of the complex permittivity standardized by the signal processing portion, thereby calculating a component concentration of the measurement subject.

Furthermore, in a configuration example of the component concentration measuring apparatus according to embodiments of the present invention, the frequency other than the frequency of the isosbestic point is a frequency at which a relaxation strength of Debye relaxation regarding hydrated water increases.

Furthermore, in a configuration example of the component concentration measuring apparatus according to embodiments of the present invention, in a case in which the measurement subject is an aqueous glucose solution, the frequency other than the frequency of the isosbestic point includes one or a plurality of frequencies in 2 to 7 GHz.

Furthermore, in a configuration example of the component concentration measuring apparatus according to embodiments of the present invention, the dielectric spectroscopy portion irradiates the measurement subject with electromagnetic waves via a dielectric spectroscopy sensor that is arranged near the measurement subject or in contact with the measurement subject, and receives electromagnetic waves from the measurement subject via the dielectric spectroscopy sensor, thereby acquiring the complex permittivity spectrum.

Furthermore, embodiments of the present invention are directed to a component concentration measuring method including: a first step of irradiating a measurement subject with electromagnetic waves, thereby acquiring a complex permittivity spectrum; a second step of standardizing an imaginary part or an imaginary part spectrum of a complex permittivity at a frequency other than a frequency of an isosbestic point of the measurement subject, using an imaginary part of a complex permittivity at the frequency of the isosbestic point, out of the complex permittivity spectrum; and a third step of applying a calibration model generated in advance from an imaginary part or an imaginary part spectrum of a complex permittivity of a sample whose component concentration is known, to the imaginary part or the imaginary part spectrum of the complex permittivity standardized in the second step, thereby calculating a component concentration of the measurement subject.

### Effects of Embodiments of the Invention

According to embodiments of the present invention, it is possible to suppress the influence of a change in the water content when measuring the component concentration of a measurement subject such as a living body, by standardizing an imaginary part or an imaginary part spectrum of a complex permittivity at a frequency other than a frequency of an isosbestic point of the measurement subject, using an imaginary part of a complex permittivity at the frequency of the isosbestic point, and thus embodiments of the invention have an effect of making it possible to measure the component concentration of a measurement subject at a high level of precision.

### BRIEF DESCRIPTION OF THE DRAWINGS

FIG. 1 is a block diagram showing the configuration of a component concentration measuring apparatus according to an embodiment of the present invention.

FIG. 2 is a block diagram showing a configuration of a dielectric spectroscopy portion of the component concentration measuring apparatus according to the embodiment of the present invention.

FIG. 3 is a block diagram showing another configuration of the dielectric spectroscopy portion of the component concentration measuring apparatus according to the embodiment of the present invention.

FIG. 4 is a flowchart illustrating the processing flow of the component concentration measuring apparatus according to the embodiment of the present invention.

FIG. 5 is a chart showing imaginary part spectra of dielectric spectroscopy of pure water and aqueous glucose solutions.

FIG. 6 is a partially enlarged view of FIG. 5.

FIG. 7 is a chart showing differential spectra of the imaginary part spectra of dielectric spectroscopy of the aqueous glucose solutions, relative to the imaginary part spectrum of pure water.

FIG. 8 is a chart showing an example of dielectric spectroscopy measurement results of glucose solutions with different water contents.

FIG. 9 is a chart showing an example of standardization according to the embodiment of the present invention.

FIG. 10 is a block diagram showing a configuration example of a computer that realizes the component concentration measuring apparatus according to the embodiment of the present invention.

### DETAILED DESCRIPTION OF ILLUSTRATIVE EMBODIMENTS

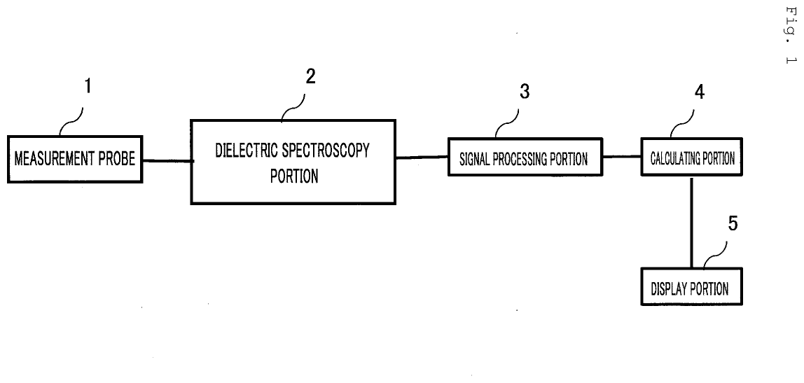

Hereinafter, an embodiment of the present invention will be described with reference to the figures. FIG. 1 is a block diagram showing the configuration of a component concentration measuring apparatus according to an embodiment of the present invention. The component concentration measuring apparatus shown in the figure includes a measurement probe 1 that is arranged near a measurement subject (not shown) or in contact with the measurement subject, a dielectric spectroscopy portion 2, a signal processing portion 3, a calculating portion 4, and a display portion 5.

The dielectric spectroscopy portion 2 is a device that irradiates a measurement subject that is a living body, a liquid, a solid, or the like with electromagnetic waves in microwave to millimeter-wave bands, and detects electromagnetic waves reflected off the measurement subject or electromagnetic waves transmitted through the measurement subject, thereby acquiring a complex permittivity spectrum (a dielectric relaxation spectrum). “Living body” is a human, an animal, a cell, or the like. If the measurement subject is a human or an animal, measurement is performed while attaching the measurement probe 1 to a portion where the measurement probe 1 can be attached with ease, such as an earlobe, an arm, a palm, a leg, a belly, or the like.

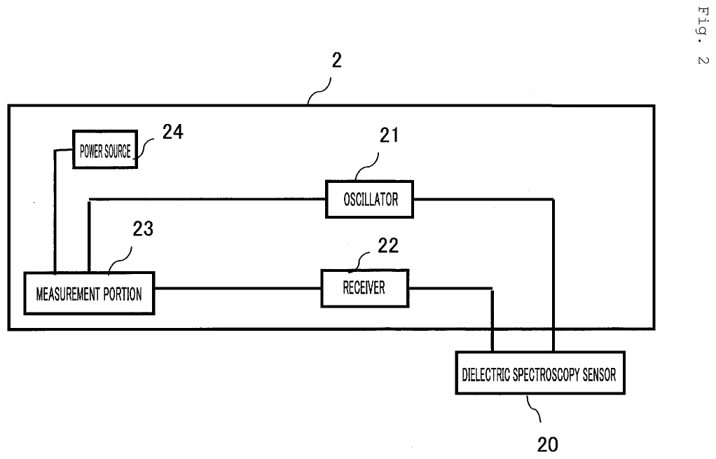

FIG. 2 is a block diagram showing the configuration of the dielectric spectroscopy portion 2. The dielectric spectroscopy portion 2 includes an oscillator 21 that supplies a signal in microwave to millimeter-wave bands to a dielectric spectroscopy sensor 20 provided in the measurement probe 1, a receiver 22 that receives electromagnetic waves reflected off a measurement subject or electromagnetic waves transmitted through the measurement subject, via the dielectric spectroscopy sensor 20, a measurement portion 23 that calculates a complex permittivity spectrum from the amplitude or the phase of the electromagnetic waves received by the receiver 22, and a power source 24.

Examples of such a dielectric spectroscopy portion 2 include a vector network analyzer (VNA) and an impedance analyzer (IA).

As the dielectric spectroscopy sensor 20, a coaxial probe, a waveguide, a microstrip line, a coplanar line, and the like can be used.

As the oscillator 21, a broadband oscillator (VCO: voltage controlled oscillator), a dielectric oscillator, a synthesizer, and the like can be used. The measurement portion 23 is constituted by a microprocessor, a micro controller unit (MCU), or the like. As the power source 24, an AC adapter, a battery, or the like is used.

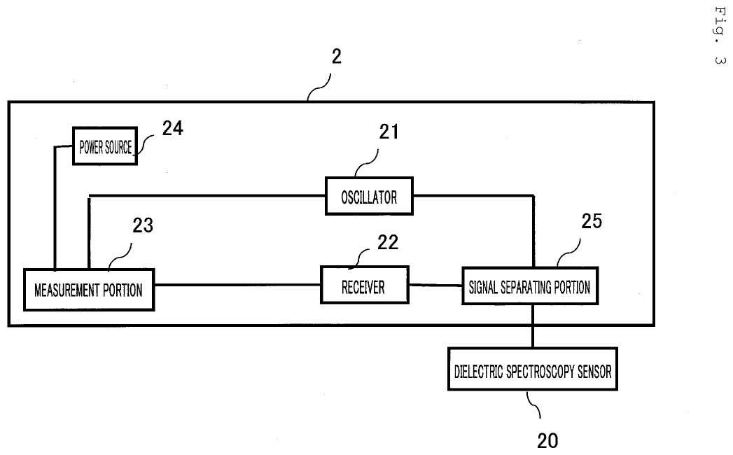

In the example shown in FIG. 2, the dielectric spectroscopy sensor 20 that independently emits and receives electromagnetic waves was described as an example. When using the dielectric spectroscopy sensor 20 that emits and receives electromagnetic waves through a common structure, it is sufficient that the dielectric spectroscopy portion 2 is provided with a signal separating portion 25 as shown in FIG. 3. The signal separating portion 25 supplies a signal from the oscillator 21 to the dielectric spectroscopy sensor 20, and outputs electromagnetic waves from the dielectric spectroscopy sensor 20 to the receiver 22. As the signal separating portion 25, a directional coupler, a circulator, and the like can be used.

The complex permittivity of a measurement subject is measured, for example, in a broadband region at 10 MHz to 70 GHz using the above-described dielectric spectroscopy portion 2.

Furthermore, instead of the dielectric spectroscopy portion 2 including a VNA or an IA, it is also possible to use a dielectric spectroscopy portion 2 including a combination of a microwave to millimeter-wave generator using two types of lasers and photo mixers, and a receiver such as a Schottky barrier diode. As the photo mixers, a PIN photodiode, an avalanche photodiode, a uni-traveling-carrier photodiode, or the like is used. As the receivers, a planar-doped barrier diode, a spectrum analyzer, a bolometer, a Golay cell, or the like may be used instead of a Schottky barrier diode. Furthermore, the free space method using a VNA and a liquid cell may be used as the permittivity measuring method. In this case, time-domain spectroscopy using a photoconductive antenna instead of a VNA or frequency-domain spectroscopy using a signal source including two types of lasers and photo mixers may be used. The dielectric spectroscopy portion 2 may be obtained by combining these plurality of methods.

The signal processing portion 3 performs pre-processing of a signal in order to improve the S/N ratio of the complex permittivity spectrum obtained by the dielectric spectroscopy portion 2. Examples of the pre-processing include processing for removing noise superimposed on a spectrum, such as averaging by measuring signals at the same frequency a plurality of times, smoothing using a moving average of a spectrum, smoothing of a spectrum using a Savitzky-Golay filter, a first derivation of a spectrum, a second derivation of a spectrum, centralization of a spectrum, scaling, multiplicative scatter correction (MSC), multiplicative scatter correction (SNV), and the like. Furthermore, the signal processing portion 3 standardizes an imaginary part or an imaginary part spectrum of the obtained complex permittivity. The standardization will be described later in detail.

The calculating portion 4 obtains the component concentration of the measurement subject, based on the imaginary part or the imaginary part spectrum of the complex permittivity standardized by the signal processing portion 3. If the standardized signal has one frequency, the calculating portion 4 performs conversion to the component concentration of the measurement subject, using a scaling factor and a bias. Furthermore, if the standardized signal has a plurality of frequencies, the calculating portion 4 obtains the component concentration of the measurement subject, using an imaginary part spectrum of the complex permittivity standardized by the signal processing portion 3, and a calibration model generated in advance from a sample whose component concentration is known.

The calibration model can be generated by irradiating a sample that is made of the same material as the measurement subject and whose component concentration is known, with electromagnetic waves in microwave to millimeter-wave bands, and detecting electromagnetic waves reflected off the sample or electromagnetic waves transmitted through the sample, thereby acquiring a complex permittivity spectrum, and subjecting the complex permittivity spectrum to multivariate analysis. In this example, a calibration model is generated through multivariate analysis, while taking a known component concentration of a sample as a response variable, and taking a complex permittivity spectrum as an explanatory variable. Examples of the multivariate analysis method include statistical methods such as multiple regression analysis, partial least squares (PLS) regression analysis, principal-component analysis, principal-component regression, logistic regression, sparse modeling, machine learning using a neural network, and analysis methods obtained by combining these methods.

The display portion 5 displays the component concentration of the measurement subject obtained as a result of calculation by the calculating portion 4. The display portion 5 may be a display apparatus such as a liquid crystal display, or may be a computer (PC) or a smartphone connected to the calculating portion 4, for example, using Bluetooth (registered trademark).

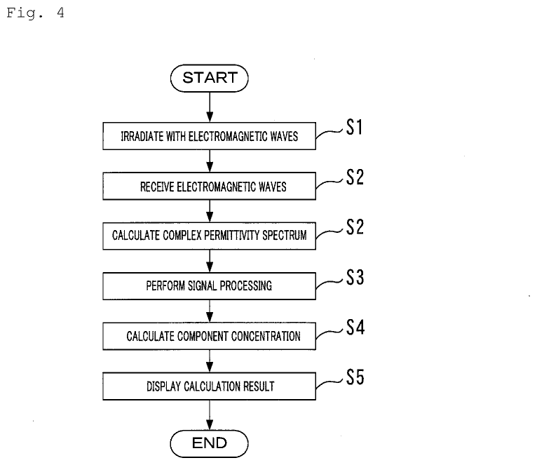

FIG. 4 is a flowchart illustrating the processing flow of the component concentration measuring apparatus. As described above, the dielectric spectroscopy portion 2 irradiates a measurement subject with electromagnetic waves via the dielectric spectroscopy sensor 20 (step S1 in FIG. 4), receives electromagnetic waves reflected off the measurement subject or electromagnetic waves transmitted through the measurement subject, via the dielectric spectroscopy sensor 20 (step S2 in FIG. 4), and calculates a complex permittivity of the measurement subject, thereby acquiring a complex permittivity spectrum (step S3 in FIG. 4).

The signal processing portion 3 performs signal processing including the above-described standardization on an imaginary part or an imaginary part spectrum of the complex permittivity (step S3 in FIG. 4).

The calculating portion 4 calculates a component concentration of the measurement subject, based on the imaginary part or the imaginary part spectrum of the complex permittivity standardized by the signal processing portion 3 (step S4 in FIG. 4), and the display portion 5 displays a result of the calculation by the calculating portion 4 (step S5 in FIG. 4).

Next, a complex permittivity spectrum that is measured by the dielectric spectroscopy portion 2 will be described. The complex permittivity spectrum obtained by the dielectric spectroscopy portion 2 is a complex number, where a real part of the complex number corresponds to a permittivity, and an imaginary part thereof corresponds to a loss of electromagnetic waves with which the measurement subject was irradiated. At this time, the complex permittivity spectrum in microwave to millimeter-wave bands is represented by Expression (i) below.

$$Formula 1$$

$${ɛ}^{*} ( ω ) - {ɛ}_{∞} = {\sum }_{n} \frac{{Δɛ}_{n}}{1 + i {ωτ}_{n}} - i \frac{σ}{{ɛ}_{0} ω} \qquad ( 1 )$$

In Expression (i), ε*(ω) is a complex permittivity of a measurement subject at each frequency ω, ε_{∞} is a static permittivity, Δεₙ is a relaxation strength of Debye relaxation, τ₀ is a relaxation time of Debye relaxation, εₒ is a permittivity of vacuum, and σ is an electrical conductivity of a measurement subject. The first term on the right side in Expression (i) is a linear combination of a Debye relaxation model. n is the number of linear combinations, and is determined by solute and the hydration number of the solute in solvent. A real part ε′(ω) and an imaginary part ε″(ω) of the complex permittivity ε*(ω) are defined in Expression (2) below.

Formula 2

ε*(ω)=ε′(ω)−iε″(ω) (2)

From the real part and the imaginary part in Expression (i) and Expression (2), ε′(ω) and ε″(ω) are represented by Expressions (3) and (4) below.

$$Formula 3$$

$$\begin{matrix}{ɛ}^{′} ( ω ) = {ɛ}_{∞} + {\sum }_{n} \frac{{Δɛ}_{n}}{1 + {( {ωτ}_{n} )}^{2}} & ( 3 ) \\ {ɛ}^{″} ( ω ) = {\sum }_{n} \frac{{Δɛ}_{n} {ωτ}_{n}}{1 + {( {ωτ}_{n} )}^{2}} + \frac{σ}{{ɛ}_{0} ω} & ( 4 )\end{matrix}$$

An imaginary part ε″(ω) of a complex permittivity represented by Expression (4) corresponds to a dielectric loss. If the measurement subject is a single component-based aqueous solution composed of molecules with a molecular weight of approximately 180, such as glucose, the complex permittivity spectrum is represented by three linear combinations as in Expression (5) below from linear combinations of a Debye relaxation model.

$$Formula 4$$

$${ɛ}^{*} ( ω ) - {ɛ}_{∞} = \frac{{Δɛ}_{s}}{1 + i {ωτ}_{s}} + \frac{{Δɛ}_{h}}{1 + i {ωτ}_{h}} + \frac{{Δɛ}_{b}}{1 + i {ωτ}_{b}} \qquad ( 5 )$$

In the expression, the subscripts s, h, and b of Δε and τ respectively mean solute, hydrated water, and bulk water. That is to say, the first term on the right side in Expression (5) is a Debye relaxation model of solute, the second term on the right side is a Debye relaxation model of hydrated water, and the third term on the right side is a Debye relaxation model of bulk water. There may be a case in which relaxation of bulk water is divided into two types of relaxation, i.e., slow relaxation involving hydrogen bonding and rapid relaxation not involving hydrogen bonding, and a complex permittivity spectrum is represented by four linear combinations. Furthermore, if the measurement subject is an aqueous solution of protein such as lysozyme or albumin, the number of Debye relaxations regarding hydrated water increases, for example, the number of Debye relaxations may be two in the case of lysozyme and approximately 4 to 5 in the case of albumin.

In this manner, the number of linear combinations of Debye relaxations increases in accordance with the number of components of a measurement subject. When the glucose concentration increases, the level of relaxation of hydrated water due to solute and glucose increases, and the level of relaxation of bulk water decreases due to exclusion of water, and thus a spectrum change in which a peak frequency is shifted is obtained.

In Expression (i), the second term on the right side represents a conduction loss. A conduction loss is a function of electrical conductivity of a measurement subject, and the electrical conductivity mainly depends on the concentration of ions in a measurement subject or the temperature of a measurement subject. If blood, a living body, or the like is taken as a measurement subject, a spectrum based on Expression (i) in which various components are mixed is acquired.

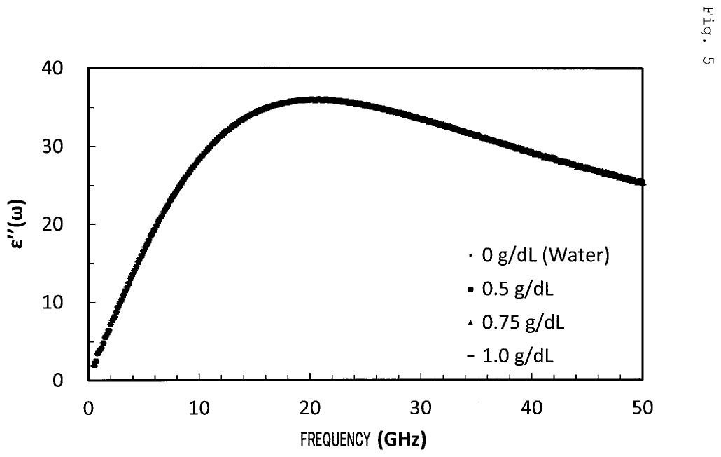

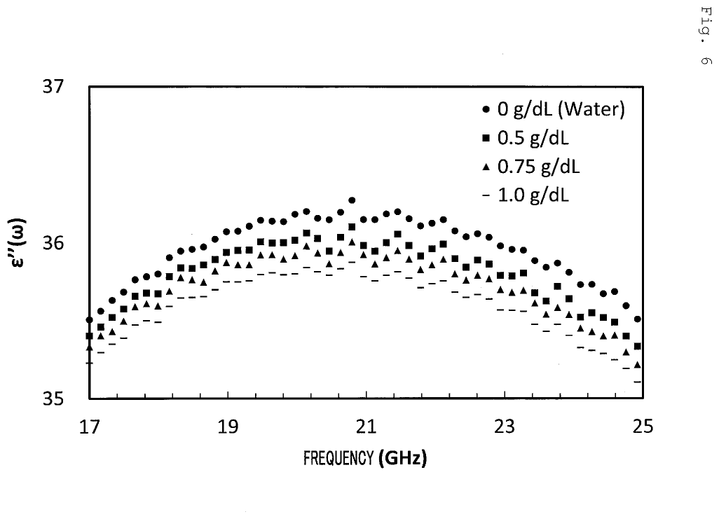

FIG. 5 shows imaginary part spectra ε″(ω) of dielectric spectroscopy of pure water (glucose concentration 0 g/dL) and aqueous glucose solutions, and FIG. 6 is an enlarged view of FIG. 5 in a range of 17 to 25 GHz. It is seen from FIGS. 5 and 6 that a complex permittivity spectrum changes depending on a glucose concentration.

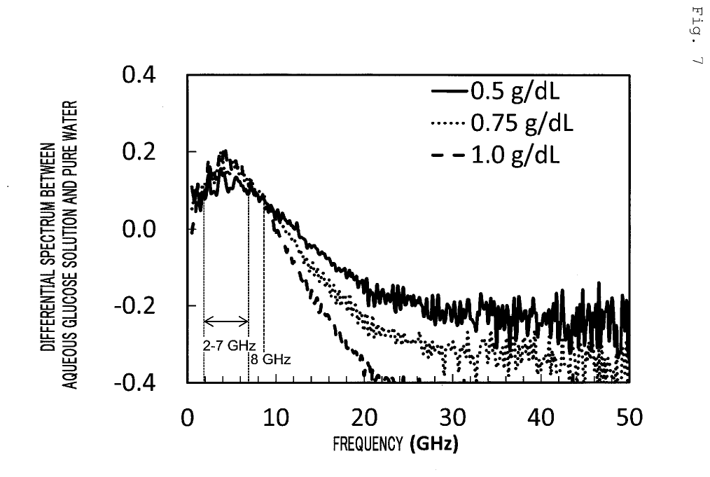

FIG. 7 shows differential spectra of the imaginary part spectra of dielectric spectroscopy of the aqueous glucose solutions, relative to the imaginary part spectrum of pure water, shown in FIGS. 5 and 6. It seems that a frequency domain in which values of a differential spectrum are positive is obtained due to an increase in the relaxation strength of solute and hydrated water, and a domain in which values of a differential spectrum are negative is obtained due to a decrease in the relaxation strength of bulk water.

Since a decrease in the relaxation strength of bulk water is caused by hydration or exclusion of water, it may occur due to a change in the concentrations of various components. When detecting a change in a spectrum unique to glucose, it seems that a change in a frequency band at 2 to 7 GHz is a change in a peak unique to glucose. Furthermore, the differential spectra takes substantially the same value regardless of the glucose concentrations at a frequency of around 8 GHz, and it seems that a frequency of around 8 GHz corresponds to the isosbestic point of glucose.

Thus, the signal processing portion 3 of this embodiment performs standardization as shown in Expression (6) below on the imaginary part ε″(ω) at one frequency or the imaginary part spectrum ε″(ω) at a plurality of frequencies, out of the complex permittivity (the real part spectrum ε′(ω) and the imaginary part spectrum ε″(ω)) acquired by the dielectric spectroscopy portion 2.

$$Formula 5$$

$${ɛ}_{std}^{″} ( ω ) = \frac{{ɛ}^{″} ( ω )}{{ɛ}^{″} ( {ω}_{std} )} \qquad ( 6 )$$

That is to say, a value obtained by dividing ε″(ω) by ε″(ω_{std}) is taken as a standardized imaginary part ε″_{std}(ω) or imaginary part spectrum ε″_{std}(ω) of complex permittivity. In the expression, ω_{std} is a frequency of an isosbestic point used for standardization, and is different between components of interest. If the measurement subject is glucose, for example, a frequency of approximately 8 GHz±1 GHz is taken as ω_{std}. ε″(ω_{std}) is an imaginary part of the complex permittivity at the isosbestic point of the measurement subject. The frequency ω that is to be standardized is any frequency other than ω_{std}, and, for example, one frequency at 2 to 7 GHz is used. In Expression (6), ε″(ω) is an imaginary part of the complex permittivity at the one frequency. If the proportion of the measurement subject is sufficiently large out of the factors of changes common to relaxation of bulk water, the change in the relaxation strength of bulk water can be used, and thus, for example, a frequency of 8 GHz or more may be taken as ω.

Furthermore, as described above, it is also possible to standardize the imaginary part spectrum ε″(ω) at a plurality of frequencies w. In this case, the measurement frequency band may be set to about 10 MHz to 70 GHz. If the measurement subject is a molecule with a large molecular weight such as protein, the measurement may be performed while lowering the lower limit of the measurement frequency to 1 kHz.

According to the standardization of this embodiment, even in an environment in which the water content changes during measurement, it is possible to prevent the level of measurement precision of component concentration from being lowered. Furthermore, according to this embodiment, an isosbestic point with the lowest dependency on the concentration of the measurement subject is used for standardization, and thus it is possible to perform measurement with a high sensitivity for a change in components of the measurement subject.

It is assumed that the approximation of an imaginary part of a complex permittivity of a measurement subject in which the water content changes can be represented by Expression (7) below.

Formula 6

ε′_{measured}(ω)=α·ϵ″_{debye}(θ)+(1−α)·ε″_{ConSt} (7)

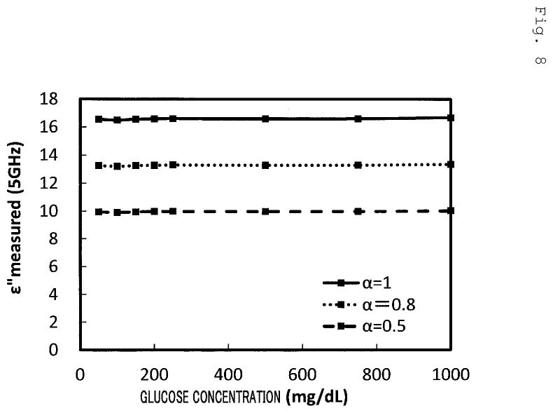

In the expression, ε″_{debye} is an imaginary part spectrum of a complex permittivity of a liquid represented by Expression (a). ε″_{const} is an imaginary part of a complex permittivity of a material other than a liquid, as measured according to a change in the water content, and is approximated as being a term that does not depend on the frequency. α is a water content, where 0≤α≤1. FIG. 8 shows calculation results of an imaginary part ε″_{measured} of a plurality of permittivities of glucose at a frequency of 5 GHz, using Expression (7), in a case in which the measurement results of the imaginary parts of the complex permittivity of aqueous glucose solutions with different concentrations are taken as ε″_{debye}, ε″_{const}=0.02, and α=1.0, 0.8, 0.5 in Expression (7).

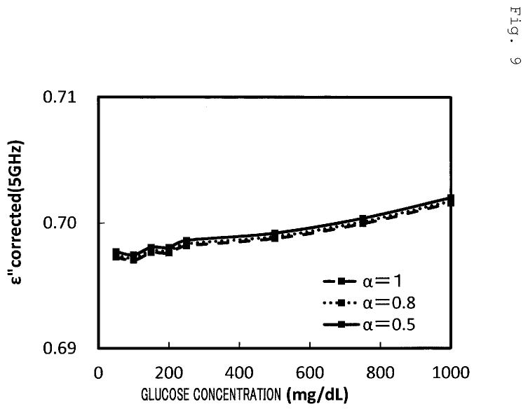

FIG. 9 shows a result regarding imaginary parts the complex ε″_{measured}(ω)) of permittivity at 5 GHz and 8 GHz calculated under similar conditions, the result being obtained using Expression (6) through standardization of the imaginary part the complex ε″_{measured}(ω) of permittivity at 5 GHz while setting ω_{std}=8 GHz, using the imaginary part ε″_{measured}(ω_{std}) of the complex permittivity at 8 GHz. It is seen from FIG. 9 that, through standardization of this embodiment, the influence of the water content is suppressed, and the calculated values are substantially the same regardless of the water content.

The calculating portion 4 of this embodiment applies a calibration model generated in advance from a sample whose component concentration is known, to the imaginary part ε″_{std}(ω) or the imaginary part spectrum ε″_{std}(ω) of the complex permittivity standardized by the signal processing portion 3, thereby calculating a component concentration of the measurement subject. Specifically, the standardized imaginary part ε″_{std}(ω) or imaginary part spectrum ε″_{std}(ω) of the complex permittivity is converted into the component concentration of the measurement subject using Expression (8) below.

Formula 7

C=A·ε″^{std}(ω)+B (8)

Expression (8) is a polynomial representing a calibration model. A is a coefficient for scaling, and B is bias. If ε″_{std}(ω) is a spectrum, the first term on the right side of Expression (8) is an inner product of the coefficient and the standardized imaginary part spectrum, and a higher level of precision can be expected through methods such as signal processing or multivariate analysis performed by the signal processing portion 3 or the calculating portion 4.

Note that, in the case of calculating the component concentration based on the imaginary part ε″_{std}(ω) of the complex permittivity at one frequency standardized by the signal processing portion 3, a calibration model generated in advance while taking the imaginary part of the complex permittivity of the sample at the same frequency as an explanatory variable, and taking a known component concentration of the sample as a response variable is used. Meanwhile, in the case of calculating the component concentration based on the imaginary part spectrum ε″_{std}(ω) at a plurality of frequencies standardized by the signal processing portion 3, a calibration model generated in advance while taking the imaginary part spectrum of the sample at the plurality of same frequencies as an explanatory variable, and taking a known component concentration of the sample as a response variable is used.

As described above, according to this embodiment, it is possible to acquire a complex permittivity spectrum of a measurement subject such as a living body, using the dielectric spectroscopy portion 2 that can measure complex permittivity in MHz to GHz bands, and to measure the component concentration of the measurement subject at a high level of precision with a simple system configuration as follows.
A drift of an imaginary part of a complex permittivity is standardized using a frequency band at two or more frequencies including a frequency corresponding to an isosbestic point of a measurement subject and a frequency at which the amount of change in the complex permittivity spectrum derived from the measurement subject is large, so that a drift of a plurality of permittivities resulting from a change in the water content or the like can be suppressed. Thus, it is possible to acquire a stable imaginary part or imaginary part spectrum of the plurality of permittivities.

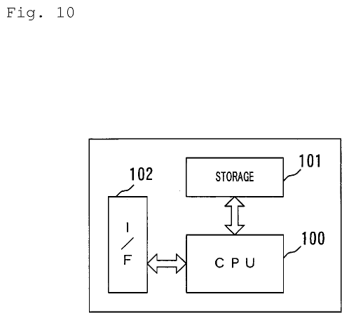

The signal processing portion 3 and the calculating portion 4 of the component concentration measuring apparatus described in this embodiment can be realized by a computer including a central processing unit (CPU), a storage, and an interface, and a program for controlling these hardware resources. FIG. 10 shows a configuration example of the computer. The computer includes a CPU 100, a storage 101, and an interface (hereinafter, abbreviated as an “I/F”) 102. The I/F 102 is connected to the dielectric spectroscopy portion 2 and the display portion 5. In this computer, a program for realizing the component concentration measuring method according to embodiments of the present invention is stored in the storage 101. The CPU 100 executes the processing described in this embodiment according to the program stored in the storage 101. Note that a computer that realizes the measurement portion 23 of the dielectric spectroscopy portion 2 may be the same as the computer that realizes the signal processing portion 3 and the calculating portion 4, or a computer that is different therefrom.

### INDUSTRIAL APPLICABILITY

The present invention can be applied to component concentration measurement using the dielectric spectroscopic technique.

### REFERENCE SIGNS LIST

1 Measurement probe
2 Dielectric spectroscopy portion
3 Signal processing portion
4 Calculating portion
5 Display portion
20 Dielectric spectroscopy sensor
21 Oscillator
22 Receiver
23 Measurement portion
24 Power source
25 Signal separating portion.

1.-8. (canceled)

9. A component concentration measuring apparatus comprising:
a dielectric spectroscopy portion that irradiates a measurement subject with electromagnetic waves to acquire a complex permittivity spectrum;
a signal processor that standardizes an imaginary part or an imaginary part spectrum of a first complex permittivity at a first frequency other than a second frequency of an isosbestic point of the measurement subject using an imaginary part of a second complex permittivity at the second frequency of the isosbestic point, wherein the first complex permittivity at the first frequency is comprised in the complex permittivity spectrum; and
a calculator that applies a calibration model generated to the imaginary part or the imaginary part spectrum of the first complex permittivity standardized by the signal processor to calculate a component concentration of the measurement subject, wherein the calibration model is generated in advance from an imaginary part or an imaginary part spectrum of a third complex permittivity of a sample with a known component concentration.

10. The component concentration measuring apparatus according to claim 9, wherein the first frequency is a frequency at which a relaxation strength of Debye relaxation regarding hydrated water increases.

11. The component concentration measuring apparatus according to claim 10, wherein the first frequency includes a frequency in a range of 2 GHz to 7 GHz when the measurement subject is an aqueous glucose solution.

12. The component concentration measuring apparatus according to claim 9, wherein the dielectric spectroscopy portion:
irradiates the measurement subject with electromagnetic waves via a dielectric spectroscopy sensor; and
receives second electromagnetic waves from the measurement subject via the dielectric spectroscopy sensor to acquire the complex permittivity spectrum.

13. The component concentration measuring apparatus according to claim 12, wherein the dielectric spectroscopy sensor is in contact with the measurement subject when the measurement subject is irradiated.

14. The component concentration measuring apparatus according to claim 12, wherein the dielectric spectroscopy sensor is spaced apart from the measurement subject when the measurement subject is irradiated.

15. A component concentration measuring method comprising:
irradiating a measurement subject with electromagnetic waves to acquire a complex permittivity spectrum;
standardizing, by a signal processor, an imaginary part or an imaginary part spectrum of a first complex permittivity at a first frequency other than a second frequency of an isosbestic point of the measurement subject using an imaginary part of a second complex permittivity at the second frequency of the isosbestic point, wherein the first complex permittivity at the first frequency is comprised in the complex permittivity spectrum; and
applying a calibration model generated to the imaginary part or the imaginary part spectrum of the first complex permittivity standardized by the signal processor to calculate a component concentration of the measurement subject, wherein the calibration model is generated in advance from an imaginary part or an imaginary part spectrum of a third complex permittivity of a sample with a known component concentration.

16. The component concentration measuring method according to claim 15, wherein the first frequency is a frequency at which a relaxation strength of Debye relaxation regarding hydrated water increases.

17. The component concentration measuring method according to claim 15, wherein the first frequency includes a frequency in a range of 2 GHz to 7 GHz when the measurement subject is an aqueous glucose solution.

18. The component concentration measuring method according to claim 15, wherein irradiating the measurement subject with the electromagnetic waves comprises:
irradiating the measurement subject via a dielectric spectroscopy sensor; and
receiving electromagnetic waves from the measurement subject via the dielectric spectroscopy sensor to acquire the complex permittivity spectrum.

19. The component concentration measuring method according to claim 18, wherein the dielectric spectroscopy sensor is in contact with the measurement subject when the measurement subject is irradiated.

20. The component concentration measuring method according to claim 18, wherein the dielectric spectroscopy sensor is spaced apart from the measurement subject when the measurement subject is irradiated.
